(19) United States
(12) Patent Application Publication
(10) Pub. No.: US 2007/0160986 A1
(43) Pub. Date: Jul. 12, 2007

(54) Method for identification of novel virulence associated genes

(76) Inventors: Ake Forsberg, Umea (SE); Sara Garbom, Umea (SE); Britt-Marie Kihlberg, Umea (SE); Hans Olof Wolf-Watz, Umea (SE)

Correspondence Address: DICKSTEIN SHAPIRO LLP, 1177 AVENUE OF THE AMERICAS (6TH AVENUE), NEW YORK, NY, 10036-2714, US

(21) Appl. No.: 10/544,989

(22) PCT Filed: Feb. 13, 2004
(86) PCT No.: PCT/SE04/00185

§ 371 Date: May 26, 2006

(30) Foreign Application Priority Data
Feb. 15, 2003 (SE) 0300413-2

(51) Int. Cl. C12Q 1/68; G06F 19/00

(52) U.S. Cl. 435/6; 702/20

(57) ABSTRACT

In a method of identifying novel virulence associated genetic information about a pathogenic microorganism including information about open reading frames (ORFs) having (a) assigned functions, (b) no assigned functions, (c) showing similarity with hypothetical proteins from other species is provided. An obligate human parasite evolutionary deprived of a substantial portion of its coding capacity is selected and genetic information about it is provided. By comparing the genetic information at least one open reading frame (ORF) common to the microorganism and the human parasite is identified, which codes for a protein (c) showing similarity with a hypothetical protein from other microorganism species. By introduction of a mutation in the ORF a mutated ORF is produced. The pathogenicity of the mutated pathogenic organism is assessed and compared with that of the corresponding non-mutated pathogenic organism.

### FIELD OF THE INVENTION

The present invention relates to a method for identification bf virulence associated genes (vag-genes). The method also relates to are virulence-promoting proteins expressed by such vag-genes, vag-genes comprising single mutations, mono- and polyclonal antibodies directed against proteins expressed by vag-genes, and methods of treating and preventing bacterial infection.

### BACKGROUND OF THE INVENTION

Antibacterial therapy is gradually becoming less effective due to the uncontrolled use of antibiotics leading to the spread of resistant bacterial strains [1]. It is therefore desirable to identify novel targets that can be used to develop antimicrobial agents having new mechanisms of action. It is evident that one important method to attack this problem will depend on the wealth of information that is now being compiled in databases via different large-scale genome projects. Today about 80 bacterial genomes including several important pathogens have been sequenced. For most bacterial genomes around a quarter of the ORFs are conserved hypothetical genes with unknown fimctions [2]. It can be predicted that among this group of ORFs protein classes will be identified, which likely could constitute suitable molecular targets for novel drugs. In line with this idea, a number of studies have already been conducted in which homologous classes of genes, from different bacteria, encoding gene products of unknown or hypothetical functions have been identified in silico and the corresponding genes have been subjected to mutagenesis in E. coli aiming at the identification of proteins essential for growth of the bacteria during in vitro conditions [3]. Thus, these proteins constitute potential targets for the development of novel antibacterial agents having a growth inhibitory effect. A number of genes have already been ascribed these desirable features [4-7].

### OBJECTS OF THE INVENTION

It is an object of the present invention to provide a method of identifying novel virulence associated genes,

It is another object of the invention to provide virulence-promoting proteins expressed by such genes and antibodies directed against these proteins

It is a further object of the present invention to provide a method of preventing or treating bacterial infection by administration of said antibodies.

Further object of the present invention will become evident by the study of the following description of the invention and preferred embodiments thereof as well as from the appended claims.

### SUMMARY OF THE INVENTION

The present invention is based on the insight that it is possible to identify classes of novel virulence factors common among several pathogenic strains that can be used as target proteins for novel antibacterial therapy. Several obligatory bacterial parasites have genome sizes that are considerable smaller when compared to free-living eubacteria. In the course of evolution these parasites have condensed their genomes and eliminated redundant genes that are not required for growth in the animal host. They have however retained a subset of genes that are essential for survival in the host during infection. Some of these “survival” genes are common for several different parasites. The present inventors consider the corresponding gene products to be amenable for attack by antibacterial agents.

In accordance with this hypothesis, obligate human parasites should contain a set of genes that are not essential for in vitro growth in rich media but that are required for survival in the animal host. Treponema pallidum contains a circular chromosome about 1 Mb in size indicating that the parasite during evolution has lost about 75% of its previous coding capacity [8]. About 53% of the ORFs have assigned functions whereas 27% of the ORFs exhibit no matches in databases. About 17% of the ORFs show similarity with hypothetical proteins from other species. Since we aimed at identifying novel “survival” genes, we analyzed the latter class of hypothetical proteins with respect to the presence of homologous proteins in three other obligatory Gram-negative (Gram−) bacterial parasites, Neisseria gonorrhoeae, Helicobacter pylori and Borrelia burgdorferi, respectively, all giving chronic diseases in humans [9]. In addition, to broaden the search and to make it more general we also included one Gram-positive (Gram+) human pathogen, Streptococcus pneumonia. This comparison resulted in the identification of 17 different genes (Virulence Associated Genes, “vag”s) that showed considerable similarity among the different pathogens including Yersinia pestis. These genes were all knocked out in Yersinia pseudotuberculosis serotype 01b and the phenotypes of these mutations were analyzed in a mouse model. In this context Y. pseudotuberculosis constitutes a good model since the wild-type causes a lethal disease in rodents allowing a rapid screen for the virulence phenotype of different mutants.

According to the present invention, five vag-genes were found to be directly involved in virulence of Y. pseudotuberculosis, since non-polar in frame mutations of these genes resulted in attenuated mutant strains. These mutants were not restricted for growth in rich medium, indicating that the corresponding gene products are not directly required for growth of the pathogen during infection of the host. Rather, these vag-proteins are considered to be involved in processes leading to the survival of the pathogen in vivo. Based on the insight that homologous proteins from other bacterial species may also be considered essential virulence determinants and the fact that the vags are only essential during infection makes them particularly suitable as molecular targets for discovery of novel anti-infective drugs. However, using anti-bacterial therapy involving chemical attenuation of the pathogen during infection without affecting growth requires that the host's immune defence clears the infectious agent. This requirement might not be met in some or the majority of cases. Therefore is so far an unproven novel strategy to cure infections and this strategy may very well involve unanticipated problems. It is also apparent that acute life threatening infections must be treated with broad-spectrum antibiotics but due to the emerging problem with bacterial resistance it is important to restrict the use of broad-spectrum antibiotics as much as possible. Therefore, there is an urgent demand to develop new antibacterial agents enrolling strategies that specifically counteract the development of resistance. Targeting gene-products directly associated with virulence will likely have this characteristic since there is a probability that mutations in the targeted genes leading to resistance will lead to attenuation of the pathogen and thus, resistance is not a selective advantage for the pathogen.

In summary, according to the present invention is disclosed a technique for identifying novel bacterial virulence factors that are effective only during in vivo growth.

In contrast to most bacterial genome-based searches that have focused on identification of essential genes to be used as potential targets for new types of antibiotic substances [3, 4, 6] the present inventors seeks to identify novel genes required for in vivo growth only. Such genes and their gene products constitute novel targets for antibiotic therapy, since they are essential for in-vivo bacterial virulence. The identification of these genes according to the invention is based on a novel bioinformatic approach.

Among a selection of six human pathogens, including one obligate intracellular bacteria, 17 conserved genes with unknown functions were identified. Among these 17 conserved vag-genes five genes (vagA, vagC, vagG, vagH and vagI) were confirmed to be directly associated with virulence, since non-polar mutations were still non-virulent. In addition, four vag-genes were contained within operons being associated with virulence. Thus, a surprisingly high proportion of the vag-genes is associated with virulence, demonstrating that the in silico screening strategy efficiently identified novel virulence associated genes and gene clusters. Not surprisingly, these genes are conserved among a large number of both Gram positive and Gram negative bacteria not included in the screen.

The common theme among the identified genes is their relation to fundamental cellular processes, such as DNA or RNA processing. Despite this type of predicted functions the in-vivo genes are non-essential for growth in vitro in a rich medium. This is surprising given the fact that most of the vags are also conserved among a large number of different bacteria and not particularly associated with pathogenic bacteria. One interesting example is vagA, which shows similarity to E. coli YchF. This protein contains the four structural motifs characteristic of GTP-binding proteins. YchF belongs to the group of 11 universally conserved bacterial GTPases [25]. Interestingly, YchF is found in all three domains of life [26]. Therefore, it was unexpected to find that both the vagA insertion mutant and the non-polar mutant of Yersinia were not affected in growth, but strongly attenuated in virulence compared to the wild-type strain. Interestingly, recent data using signature-tagged mutagenesis showed that the vagA/ychF homolog in Neisseria meningitis is required for virulence in an infant rat model [27]. Thus, it seems likely that YchF homologs are virulence associated proteins in several pathogens, supporting the hypothesis of the invention that Vags are common virulence associated proteins.

The gene vagI shows similarity to the E. coli conserved hypothetical gene yebC. In Y. pseudotuberculosis vagI is the third gene in what might be an operon consisting of six genes. However, the region is not particularly conserved between different bacteria with the exception of Yersinia and E. coli. This region differs also slightly when these two bacteria are compared. The non-polar vagI mutant secreted normal amounts of Yops, showed cytotoxicity towards HeLa cells, but was strongly attenuated, demonstrating that vagI is an essential virulence determinant.

The vagC homolog in E. coli, rluD, is one of the 10 known putative pseudouridine synthases of this bacteria. Pseudouridine is the most abundant and was the first modified nucleoside to be discovered in RNA [28]. Pseudouridine synthases catalyze the isomerization of specific uridines in cellular RNAs to pseudouridines and in E. coli is RluD involved in the biosynthesis of three closely located pseudouridines (psi) in 23S ribosomal RNA. A RluD deficient strain of E. coli is blocked in 23S rRNA psi formation and is growth restricted [22]. Moreover, recent data indicate that RluD in E. coli may have two different functions, since a rluD point mutant unable to form specific pseudouridines in E. coli 23S rRNA restores normal growth in a RluD null mutant [29]. Surprisingly, neither the insertion mutant nor the in-frame deletion mutant of vagC in Y. pseudotuberculosis was impaired for growth not even at competitive conditions with the wild-type strain. A vagC null mutant showed no defect in Yop secretion or cytotoxicity, still the mutants were attenuated.

Interestingly, one gene that was included in the “control” group”, miaA, also encodes a RNA modifying protein. MiaA is one of three genes required to make the modified ms²i⁶A nucleoside in tRNA. MiaA has previously been shown to be involved in virulence in Shigella, involving a mechanism affecting the expression of the transcriptional activator VirF [30]. VirF is a regulatory protein that positively regulates a Type III secretion system of Shigella. However, no such effect was noticed in Yersinia.

Although the vagG mutants were attenuated they expressed normal amounts of Yops, was as cytotoxic as the wild-type strain to HeLa cells and grow normally in rich media. The gene vagG shows homology to the conserved hypothetical gene smf of E. coli. In addition, three of four genes downstream of smf are conserved hypothetical genes and interestingly the last gene of the operon shows similarity to the E. coli shikimate 5-dehydrogenase gene aroE [31] (Karudapuram-95). The smf homologs in several bacteria, such as S. pneumonia, H. influenzae, H. pylori and B. subtilis have been implicated in the uptake of DNA in these natural competent bacteria [32-35]. However, it is not known if Smf is associated with virulence in these bacterial pathogens. Interestingly, recent results implicate a role of the smf-aroE region in the virulence in Neisseria meningitis.

Only one gene with a predicted function in protein translation was found that also was associated with virulence in Yersinia. The gene product VagH shows similarity to E. coli HemK, that recently was shown to be a N(5)-glutamine methyltransferase that modifies peptide release factors [36, 37]. The hemK gene is widespread in both Gram-negative and Gram-positive bacteria, and has been maintained in the small genomes of several obligate intracellular parasites. Mutation of vagH did not affect the growth of this bacterial strain in rich media. However, both the polar and the non-polar mutant of vagH expressed and secreted a reduced amount of Yops, but despite this defect the vagH mutant strain was still cytotoxic towards HeLa cells albeit the effect was delayed. Both in E. coli and in Yersinia the hemK gene is located just downstream of prfA (the gene encoding release factor 1 (RF1)). HemK methylates both RF1 and RF2 in vitro. However, in the case of E. coil K12 in vitro N⁵-methylation of the glutamine residue is important for the activity of RF2 [36], but contributes little to the activity of RF1 [38]. Deletion of hemk in E. coli K12 resulted in a severe growth defect in rich media, which could be reversed either by transcomplementation of hemK or by a suppressor mutation in RF2 [36, 37]. However, E. coli K12 was shown to have an unusual variant of RF2, which is more dependent on a functional HemK than RF2 in other strains of E. coli. Thus, the physiological role of HemK in other bacteria remains to be clarified. Likewise, the role for VagH/HemK in the expression and secretion of Yops remains to be clarified.

Thus, according to the present invention is disclosed a method for identifying novel virulence associated genes, comprising: selecting a pathogenic microorganism; providing genetic information about said pathogenic microorganism comprising information about open reading frames (ORFs) having (a) assigned functions, (b) no assigned functions, (c) showing similarity with hypothetical proteins from other species; selecting an obligate human parasite evolutionary deprived of a substantial portion of its coding capacity; providing genetic information about said human parasite; comparing the genetic information provided for said pathogenic microorganism with that provided for said obligate human parasite, thereby identifying at least one open reading frame (ORF) common to both coding for a protein (c) showing similarity with a hypothetical protein from other microorganism species; introducing a mutation in said at least one ORF of said pathogenic organism to produce a mutated ORF; assessing the pathogenicity of said mutated pathogenic organism; comparing the pathogenicity of said mutated pathogenic organism with that of the corresponding non-mutated pathogenic organism. Preferably the substantial portion of coding capacity is 50% or more, in particular about 75%.

It is preferred for the mutation to be an insertion mutation or a deletion mutation. The aforementioned protein (c) is preferably capable of substantially promoting in vivo growth of the microorganism by which it is expressed but is not essential, preferably protein (c) is coded by.

According to a first preferred aspect of the invention the vag-gene is selected from vagA, vagC, vagG. vagH, and vagI.

According to a second preferred aspect of the invention, the obligate human parasite is selected from Yersinia pestis, Treponema pallidum, Neisseria gonorrhoeae, Helicobacter pylori, and Borrelia burgdorferi. Preferably the obligate human parasite is evolutionary closely related to the respective pathogenic microorganism.

According to the present invention is also disclosed a virulence-promoting protein expressed by a vag-gene conserved in an obligate parasite, such as one of the parasites identified above, preferably a vag-gene selected from vagA, vagC, vagG. vagH, and vagI. Preferably the virulence-promoting protein comprises a single insertion or deletion mutation.

According to the present invention is furthermore disclosed an antibody directed against the virulence-promoting protein expressed by a vag-gene conserved in an obligate parasite, such as one of the parasites identified above, preferably a vag-gene selected from vagA, vagC, vagG. vagH, and vagI.

According to the present invention is additionally disclosed a monoclonal antibody directed against the virulence-promoting protein expressed by a vag-gene conserved in an obligate parasite, such as one of the parasites identified above, preferably a vag-gene selected from vagA, vagC, vagG. vagH, and vagI.

The present invention also comprises a method for treating bacterial infection comprising administrating to a patient in need an effective amount a monoclonal antibody directed against the virulence-promoting protein expressed by a vag-gene conserved in an obligate parasite, such as one of the parasites identified above, preferably a vag-gene selected from vagA, vagC, vagG. vagH, and vagI.

According to a third preferred aspect of the invention is disclosed chemical agent, in particular an antibiotic, directed against a vag-gene conserved in an obligate parasite, such as one of the parasites identified above, preferably a vag-gene selected from vagA, vagC, vagG. vagH, and vagI.

According to a fourth preferred aspect of the invention is disclosed a microorganism virulence reducing vaccine prepared from an attenuated vag-mutant strain, and the use of such strain as a vaccine carrier of heterologous antigens.

According to the invention is also disclosed a method of treating or preventing an infection in a patient, comprising the administration of an effective amount of a chemical agent, in particular an antibiotic, directed against a vag-gene conserved in an obligate parasite, such as one of the parasites identified above, preferably a vag-gene selected from vagA, vagC, vagG. vagH, and vagI.

According to a fifth preferred aspect of the invention is disclosed a method of preventing an infection comprising the administration an attenuated vag-mutant strain as a vaccine carrier of heterologous antigens.

According to a sixth preferred aspect of the invention is disclosed a method of treating bacterial infection in a patient, comprising the administration of an effective amount of antibody, in particular a monoclonal antibody, directed against the virulence-promoting protein expressed by a vag-gene conserved in an obligate parasite, such as one of the parasites identified above, preferably a vag-gene selected from vagA, vagC, vagG. vagH, and vagI.

### DESCRIPTION OF PREFERRED EMBODIMENTS

Materials and Methods

Bioinformatics. For all the comparisons on protein level blastp was used (ftp.ncbi.nlm.nih.gov) [39]. The cut-off e-value in all of the comparisons was set to 10⁻⁷ and proteins with a lower e-value were considered as hits. Treponema pallidum conserved hypothetical ORFs and ORFs with unknown function were downloaded at http://www.tigr.org and compared to the proteome of Yersinia pestis retrieved from http://www.sanger.ac.uk. The protein sequences (73 ORFs) from Yersinia pestis were then compared to four other bacterial genomes also downloaded from http://www.tigr.org; Helicobacter pylori 2669, Borrelia burgdorferi and Streptococcus pneumoniae Tigr4. The comparison to the unfinished genome sequence of Neisseria gonorrhoeae was performed at http://www.ncbi.nlm.nih.gov/cgi-bin/Entrez/genom table cgi using tblastn. The same cut-off was used in these comparisons. ORFs (17) with homologues in all six genomes were compared to the nr (non-redundant) database and to the human proteome downloaded from http://www.ncbi.nlm.nih.gov in order to identify a putative function and to evaluate the level of conservation. Genome references in the NCBI database genbank (Benson, D A et al., Nucleics Acid Res 2002. 30(1):17-20) useful in this invention are:
NC_{—}003143=Yersinia pestis CO92
AE000520=Treponema pallidum
AD000783=Borrelia burgdorferi
AE000511=Helicobacter pylori 26695
AE007317=Streptococcus pneumoniae R6.

In regard of Neisseria gonorrhoeae, the genome of which has not yet been fully sequenced, useful information was retrieved from ref. 39, tblastn being used in this instance.

Bacterial strains, and media. The Y. pseudotuberculosis strain IP32953 used in this study was obtained from E. Carniel at the Pasteur Institute in France. This strain was grown on plates containing 10 μg/ml nalidixin in order to select for nalidixin resistant clones. One resistant clone (IP32953Nal) was used in the all of the experiments and is referred to as wild-type. E. coli strain S17-1□pir was used for conjugative transfer of the recombinant plasmids for mutagenesis of Y. pseudotuberculosis. E. coli strains were grown in LB broth or on LB agar plates. Yersinia strains were grown in LB or brain-heart infusion (BHI; Oxoid). BHI was supplemented with 5 mM EGTA and 20 mM MgCl₂ (BHI⁻). For solid media, Yersinia selective agar base (YSA; Difco) was used. Antibiotics were used at the following concentrations; nalidixin (10 μg ml⁻¹), chloramphenicol (20 μg ml⁻¹) and carbenicillin/ampicillin (100 μg ml⁻¹).

Preparation of plasmid DNA, restriction enzyme digests, ligations and transformations into E. coli were performed essentially as described by [Sambrooks, 1989 #1319]. DNA fragments were purified from agarose gels using Ultrafree-DNA (Amicon, USA) according to the manufacturer's instructions.

Mutagenesis strategy: Insertion mutants. The insertion mutants were created as described elsewhere [40] with the exception that the silicide vector pNQ705 [18] was used in this case.

Mutagenesis strategy: Deletion mutants. All of the deletion mutants were constructed in the same way by allelic exchange as described by Milton et al [21]. The fifteen N-terminal and fifteen C-terminal amino acids were not deleted.

Growth conditions and Yop secretion and expression analysis. Yop secretion and expression analysis was carried out as described previously by Petersson et al. [41] with the exception of not adding Triton X-100 to the cultivation media.

Cytotoxicity assay. The cultivation and infection of HeLa cells has been described in detail previously by Rosqvist et al. [42].

### EXAMPLE 1

Oral infections. Bacteria were grown over night in Luria Broth at 26 degrees with shaking. A 100 ml overnight culture was centrifuged and re-suspended in 50 ml of sterilized tap water. C59 black/6 female mice were starved for water over night and challenged with the bacteria re-suspended in water at different concentrations (5×10⁹ cfu/ml, 5×10⁸ cfu/ml for the insertion mutants and also 5×10⁷ cfu/ml for the deletion mutants). Each cage contained three mice and the mice were allowed to feed on 50 ml of the bacteria for 8 hours, the mice usually consumed approximately 5 ml of the bacterial suspension. In order to calculate LD₅₀ the concentration of the bacteria fed to the mice were measured by viable count.

### EXAMPLE 2

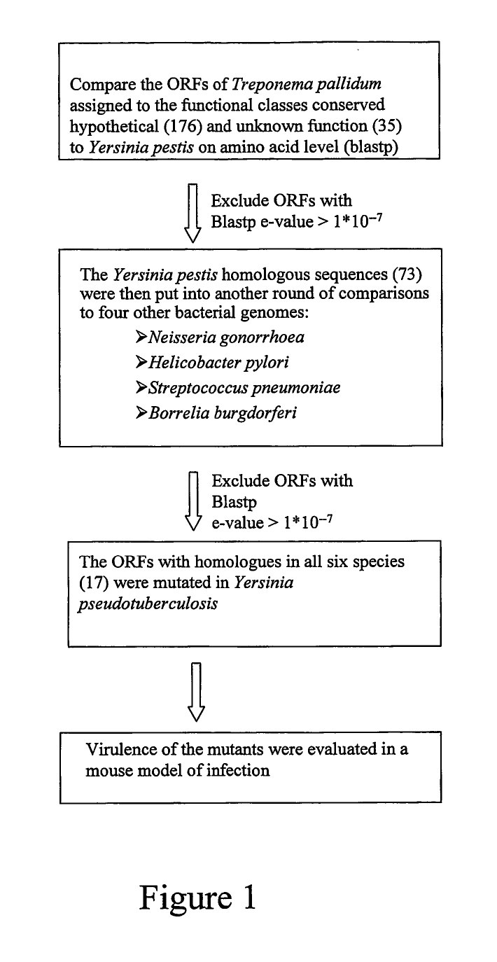

Identification of genes with an unknown function conserved among a selection of human pathogens giving chronic infections. To identify novel hitherto not characterised potential virulence associated genes Treponema pallidum [8, 10] were chosen for homology comparison to five other human pathogens (FIG. 1). At the time of initiating the study there were 176 conserved hypothetical genes and 35 genes with an unknown function in T. pallidum. These 211 ORFs from T. pallidum were first compared to the complete genome of Yersinia pestis, since this obligate pathogen seems to be in the process of undergoing adaptation to a new lifecycle that include the human host [11]. Y. pestis is closely related to the strain Y. pseudotuberculosis, and it has been proposed that Y. pestis is a clone that evolved from Y. pseudotuberculosis serotype O:1 for about 1,500-20,000 years ago [12]. Therefore, the well established Yersinia pseudotuberculosis animal model was chosen to evaluate whether the identified genes were associated with virulence. The selection criterion was chosen to identify genes with significant similarity on the protein level (BLASTP E-value<10⁻⁷). Out of the original 211 selected ORFs in the T. pallidum genome, 73 remained after comparison with the Y. pestis genome. These 73 ORFs were thereafter compared to the genomes of the four other selected human pathogens preferentially giving chronic infections; Neisseria gonorrhoeae, Helicobacter pylori, Borrelia burgdorferi and Streptococcus pneumonia [9] (FIG. 1). This comparison resulted in the identification of 17 conserved ORFs, herein named as virulence associated genes (vags) 1-17 (Tab. 1). Interestingly, as judged from comparison to the NCBI non-redundant database all vags exhibited homologues sequences in at least 35 other microorganisms, including the two other Yersinia species pathogenic for humans Yersinia pseudotuberculosis and Yersinia enterocolitica, as well as E. coli (data not shown), demonstrating that the identified Vags are highly conserved among many bacteria. A comparison against the first draft of the human genome showed that 9 of the identified 17 vags also exhibited similarity to human genes (Tab. 1).

To evaluate the strategy used for selection of potential virulence genes, a number of genes with known activity in vivo were selected and compared to the same set of bacteria described above. A collection of 99 genes with an in vivo activity demonstrated by infection with signature tagged mutants (STM) [13-16] or by selected capture of transcribed sequences (SCOTS) [17] were selected. Many of these genes are also known to be virulence associated. Therefore, for simplicity we here choose to name these genes Vir for virulence genes, despite the fact that not all of these genes have been verified to be virulence associated. Gene comparison resulted in the identification of 5 genes that were conserved among the selected human pathogens (Tab. 1). These five genes were also found to be highly conserved among many organisms, including E. coli, but they did not have any obvious human homologues.

### EXAMPLE 3

Targeted mutations of the identified Vags and analysis of the resulting virulence phenotypes. To analyse the virulence phenotypes associated with the identified vags, mutants were created in the Y. pseudotuberculosis serotype O:1 strain IP32953. Insertion mutagenesis by a single crossover event using the silicide plasmid pNQ705 [18] were used in the first round of screening for virulence phenotypes of the identified 17 vag genes. With this strategy mutants that grow in a rich medium could be constructed for 14 of the selected vag genes. The remaining three vag genes that could not be mutated are most likely essential for growth in rich media (Tab. 1). Most of the insertion mutants had the same growth rate as the wild-type strain. One exception was the mutant vagE that apart from showing a slightly reduced growth rate also exhibited an elongated filamentous form of the bacteria, especially when incubated at 37° C. The 14 insertion mutants were analysed for their ability to express and secrete known Yersinia virulence effectors called Yops (Yersinia outer proteins) as well as their ability to induce a cytotoxic response in infected HeLa cells [19] (Tab. 1). The cytotoxicity of Yersinia for HeLa cells is known to be dependent on the translocation of the Yops into the eukaryotic cell by a Type III secretion system (TTSS) [20]. All mutants expressed Yops, but vagH expressed as well as secreted a reduced amount of Yops compared to the wild-type strain. Additionally, two mutants (vagF and vagI) demonstrated a defect in their ability to secrete Yops into the growth media (Tab. 1). However, this reduced ability to secrete Yops had no or only a minor effect on the ability of the mutants to induce a cytotoxic response in infected HeLa cells. One exception was vagH, which did result in a much slower cytotoxic response in infected HeLa cells (Tab. 1). Thus, 10 of the 14 mutants showed wild-type phenotype with respect to Yop secretion and cytotoxicity towards HeLa cells, whereas the remaining 4 mutants showed a reduced ability to secrete Yops and/or a slightly delayed cytotoxicity. Similarly, insertion mutations were generated in the five conserved vir-genes that where selected from the collection of genes known to be essential for growth in vivo. All these mutants behaved as the wild-type strain with respect to growth rate, Yop secretion and cytotoxicity towards HeLa cells (Tab. 2).

The insertion mutants were thereafter screened for virulence in the mouse infection model. The oral route of infection was chosen for the first round of screening since this peripheral route of infection is likely to engage several levels of host defenses. Oral infection of the 14 vag mutants showed that 9 (63%) were attenuated in virulence, whereas the remaining 5 mutants were as virulent as the wild-type strain (Table 1). Insertional mutagenesis of the five vir-genes resulted in three (60%) attenuated mutants, whereas the remaining two mutants were as virulent as the wild-type strain (Table 2). The method according to the invention for identifying virulence genes was found to be as efficient as the classical approach.

Analysis of the DNA regions surrounding the identified vag genes revealed that most of these genes are contained within operons. Therefore, in frame deletions of the vags were made to verify that the virulence phenotype was due to the identified vag and not due to polar effects on downstream genes. The deletion mutants were constructed by using the sliicide plasmid pDM4 [21] allowing a double crossover event to occur. Deletion mutants could be constructed for eight of the nine virulence associated genes. A non-polar deletion mutant in vagD could not be constructed despite the fact that the in-frame deletion construct could recombinate with homologous sequences both upstream and downstream of the gene with a similar frequency. Thus, there seem to be a selective advantage for Yersinia to maintain vagD. All the mutants were wild-type phenotypes regarding growth in rich media. However, vagC show similarity to the E. coli ribosomal large subunit pseudouridine synthase D (rluD) that is essential for growth of E. coli [22]. Even though it has been shown in E. coli that deletion of some of the pseudouridine synthase genes individually have no effect on exponential growth in rich media [23], some pseudouridine synthases can confer a growth disadvantage compared to the wild-type strain when grown under competitive conditions [24]. Therefore, the mutant vagC was analysed for its ability to grow under competitive conditions together with the wild-type strain. This showed that the vagC deletion mutant of Yersinia grow as well as the wild-type strain also under competitive conditions (data not shown). Also Yop secretion and cytotoxicity towards HeLa cells were similar to the wild-type strain except for the mutant vagH, that was severely affected in its ability to both secrete and express Yops. Surprisingly, the vagH mutant was still cytotoxic towards HeLa cells, but the effect was more than two hours delayed (Tab. 3). By using the oral route of infection five of the eight vag-deletion mutants (vagA, vagC, vagG, vagH and vagI), where shown to be attenuated in virulence when compared to the wild-type strain, whereas the remaining three vag-deletion mutants (vagB, vagE and vagF) were as virulent as the wild-type strain (Table 3). Similarly, the deletion mutants made in the three conserved vir-genes (phAc, abc and miaA) all behaved as the wild-type strain regarding Yop expression and cytotoxicity towards HeLa cells and were found to be attenuated when mice were infected via the oral route (Table 3). These results show that vagA. vagC, vagG, vagH and vagI encode products that are essential in virulence, whereas vagB, vagE and vagF are not directly involved in virulence, but are contained within operons involved in virulence. Interestingly, the product of vagH somehow affects the expression of Yops, which are known to be essential virulence factors.

### REFERENCES

1. Hand, W. L., Current challenges in antibiotic resistance. Adolesc Med, 2000. 11(2): p. 427-38.
2. Fraser, C. M. et al., Microbial genome sequencing Nature, 2000. 406(6797): p. 799-803.
3. Chalker, A. et al., Rational identification of new antibacterial drug targets that are essential for viability using a genomics-based approach. Pharmacol Ther, 2002. 95(1): p. 1.
4. Arigoni, F. et al., A genome-based approach for the identification of essential bacterial genes. Nat Biotechnol, 1998. 16(9): p. 851-6.
5. Bruccoleri, R. E. et al., Concoradance analysis of microbial genomes. Nucleic Acids Res, 1998. 26(19): p. 4482-86.
6. Glass, J. I. et. al., Streptococcus pneumoniae as a genomics platform for broad-spectrum antibiotic discovery. Curr Opin Microbiol, 2002. 5(3): p. 338-42.
7. Thanassi, J. A., et al., Identification of 113 conserved essential genes using a high-throughput gene disruption system in Streptococcus pneumoniae. Nucleic Acids Res, 2002. 30(14): p. 3152-62.
8. Fraser, C. et al., Complete genome sequence of Treponema pallidum, the syphilis spirochete. Science, 1998. 281(5375): p. 375-388.
9. Salyers, A. A. et. al., Bacterial Pathogenesis a molecular approach. 2nd ed. ed. 2002, Washington D.C.: ASM Press.
10. Radolf, J. D. et al., Treponema pallidum: doing a remarkable job with what it's got. Trends in Microbiol., 1999. 7(1): p. 7-9.
11. Parkhill, J., et al., Genome sequence of Yersinia pestis, the causative agent of plague. Nature, 2001. 413(6855): p. 523-7.
12. Achtman, M. et al., Yersinia pestis, the cause of plague, is a recently emerged clone of Yersinia pseudotuberculosis. Proc Natl Acad Sci USA, 1999. 96(24): p. 14043-8.
13. Chiang, S. L. et al., Use of signature-tagged transposon mutagenesis to identify Vibrio cholerae genes critical for colonization. Mol Microbiol, 1998. 27(4): p. 797-805.
14. Hensel, M., et al., Simultaneous identification of bacterial virulence genes by negative selection. Science, 1995. 269(5222): p. 400-3.
15. Darwin, A. J. et al., Identification of Yersinia enterocolitica genes affecting survival in an animal host using signature-tagged transposon mutagenesis. Mol Microbiol, 1999. 32(1): p. 51-62.
16. Tsolis, R. M., et al., Identification of a putative Salmonella enterica serotype typhimurium host range factor with homology to IpaH and YopM by signature-tagged mutagenesis. Infect Immun, 1999. 67(12): p. 6385-93.
17. Graham, J. E. et al., Identification of Mycobacterium tuberculosis RNAs synthesized in response to phagocytosis by human macrophages by selective capture of transcribed sequences (SCOTS). Proc Natl Acad Sci USA, 1999. 96(20): p. 11554-9.
18. Norqvist, A. et al., Characterization of a novel chromosomal virulence locus involved in expression of a major surface flagellar sheath antigen of the fish pathogen Vibrio Anguillarum. Infect Immun, 1993. 61: p. 2434-2444.
19. Comelis, G. R. et al., The Yersinia virulon: A bacterial system for subverting eukaryotic cells. Mol. Microbiol., 1997. 23: p. 861-867.
20. Rosqvist, R. et al., Target cell contact triggers expression and polarized transfer of Yersinia YopE cytotoxin into mammalian cells. EMBO J, 1994. 13: p. 964-972.
21. Milton, D. L., et al., Flagellin A is essential for the virulence of Vibrio anguillarum. J Bacteriol, 1996. 178(5): p. 1310-9.
22. Raychaudhuri, S., et al., A pseudouridine synthase required for the formation of two universally conserved pseudouridines in ribosomal RNA is essential for normal growth of Escherichia coli. Rna, 1998. 4(11): p. 1407-17.
23. Del Campo et al., Identification and site of action of the remaining four putative pseudouridine synthases in Escherichia coli. Rna, 2001. 7(11): p. 1603-15.
24. Gutgsell, N., et al., Deletion of the Escherichia coli pseudouridine synthase gene truB blocks formation of pseudouridine 55 in tRNA in vivo, does not affect exponential growth, but confers a strong selective disadvantage in competition with wild-type cells. Rna, 2000. 6(12): p. 1870-81.
25. Caldon, C. E. et al., Evolution of a molecular switch: universal bacterial GTPases regulate ribosome function. Mol Microbiol, 2001. 41(2): p. 289-97.
26. Mittenhuber, G., Comparative genomics of prokaryotic GTP-binding proteins (the Era, Obg, EngA, ThdF (TrmE), YchF and YihA families) and their relationship to eukaryotic GTP-binding proteins (the DRG, ARF, RAB, RAN, RAS and RHO families). J Mol Microbiol Biotechnol, 2001. 3(1): p. 21-35.
27. Sun, Y. H., et al., Functional genomics of Neisseria meningitidis pathogenesis. Nat Med, 2000. 6(11): p. 1269-73.
28. Charette, M. et al., Pseudouridine in RNA: what, where, how, and why. IUBMB Life, 2000. 49(5): p. 341-51.
29. Gutgsell, N. S., et al., A second function for pseudouridine synthases: A point mutant of RluD unable to form pseudouridines 1911, 1915, and 1917 in Escherichia coli 23S ribosomal RNA restores normal growth to an RluD-minus strain, Rna, 2001. 7(7): p. 990-8.
30. Durand, J. M., et al., Transfer RNA modification, temperature and DNA superhelicity have a common target in the regulatory network of the virulence of Shigella flexneri: the expression of the virF gene. Mol Microbiol, 2000. 35(4): p. 924-935.
31. Anton, I. A. et al., Sequencing and overexpression of the Escherichia coli aroE gene encoding shikimate dehydrogenase. Biochem J, 1988, 249(2): p. 319-26.
32. Karudapuram, S. et al., DNA sequence and characterization of Haemophilus influenzae dprA+, a gene required for chromosomal but not plasmid DNA transformation. J Bacteriol, 1995. 177(11): p. 3235-40.
33. Ando, T., et al., HP0333, a member of the dprA family, is involved in natural transformation in Helicobacter pylori. J Bacteriol, 1999. 181(18): p. 5572-80.
34. Campbell, E. A., et al., A competence regulon in Streptococcus pneumoniae revealed by genomic analysis. Mol Microbiol, 1998. 27(5): p. 929-39.
35. Ogura, M., et al., Whole-genome analysis of genes regulated by the Bacillus subtilis competence transcription factor ComK. J Bacteriol, 2002. 184(9): p. 2344-51.
36. Heurgue-Hamard, V., et al., The hemK gene in Escherichia coli encodes the N(5)-glutamine methyltransferase that modifies peptide release factors. Embo J, 2002. 21(4): p. 769-78.
37. Nakahigashi, K., et al., HemK, a class of protein methyl transferase with similarity to DNA methyl transferases, methylates polypeptide chain release factors, and hemK knockout induces defects in translational termination. Proc Natl Acad Sci USA, 2002. 99(3): p. 1473-8.
38. Dincbas-Renqvist, V., et al., A post-translational modification in the GGQ motif of RF2 from Escherichia coli stimulates termination of translation. Embo J, 2000. 19(24): p. 6900-7.
39. Altschul, S. F. et al., Gapped BLAST and PSI-BLAST: a new generation of protein database search programs. Nucleic Acids Res, 1997. 25(17): p. 3389-402.
40. Rimpilainen, M. et al., A novel protein, LcrQ, involved in the low-calcium response of Yersinia pseudotuberculosis shows extensive homology to YopH. J Bacteriol, 1992. 174(10): p. 3355-63.
41. Pettersson, J., et al., The V-antigen of Yersinia is surface exposed before target cell contact and involved in virulence protein translocation. Mol Microbiol, 1999. 32(5): p. 961-76.

42. Rosqvist, R. et al., The cytotoxic protein YopE of Yersinia obstructs the primary host defence. Mol Microbiol, 1990. 4: p. 657-667.

TABLE 1
Insertion mutations of the identification 17 conserved ORFs with an unknown or hypothetical function.

| | Y. pestis | | | | | Growth, | | |
|---|---|---|---|---|---|---|---|---|
| | Sanger | E. coli | | | | rich media 26/37 | Virulence | Human |
| Name | CDS | homolog | Predicted function¹ | Yop secretion | Cytotoxicity | degrees | (oral infection) | homologue |
| VAG1 | YPO2010 | YchF | GTPase | +++ | +++ | +++/+++ | Attenuated | YES |
| VAG2 | YPO3269 | YfiF | RNA methyltransferase | +++ | +++ | +++/+++ | Attenuated | YES |
| VAG3 | YPO3277 | RluD | RNA psuedouridine synthase | +++ | +++ | +++/+++ | Attenuated | YES |
| VAG4 | YPO1062 | MesJ | Cell division | +++ | +++ | +++/+++ | Attenuated | |
| VAG5 | YPO0547 | Yabc/MraW | Methyltransferase | +++ | +++(delayed) | ++/+ | Attenuated | YES |
| VAG6 | YPO2213 | YciL | RNA psuedouridine synthase | + | +++ | +++/++ | Attenuated | |
| VAG7 | YPO0243 | Smf | DNA uptake | +++ | +++ | +++/+++ | Attenuated | |
| VAG8 | YPO2018 | HemK | methyltransferase | + | ++ | ++/++ | Attenuated | YES |
| VAG9 | YPO2055 | YebC | Unknown | − | +++(delayed) | +++/+++ | Attenuated | YES |
| VAG10 | YPO1604 | YceG | Catabolism/anabolism of | +++ | +++ | +++/+++ | Virulent | |
| | | | chorismate | | | | | |
| VAG11 | YPO2607 | NadD | Nucleotidyltransferase | +++ | +++ | ++/++ | Virulent | |
| VAG12 | YPO3547 | YraL | Methyltransferase | +++ | +++ | +++/+++ | Virulent | |
| VAG13 | YPO1607 | YcfH | Metal dependent hydrolase | +++ | +++ | +++/+++ | Virulent | YES |
| VAG14 | YPO1106 | YfjB | GTPase | +++ | +++ | +++/+++ | Virulent | YES |
| VAG15 | YPO3816 | YhhF | Methyltransferase | ND² | ND² | ND² | ND² | |
| VAG16 | YPO1049 | UppS | Undecaprenyl pyrophosphate | ND² | ND² | ND² | ND² | YES |
| | | | synthetase | | | | | |
| VAG17 | YPO2875 | EngA | GTPase | ND² | ND² | ND² | ND² | |

¹Putative function as assigned to the homologous gene in E. coli.
²ND = Not Determined, not able to obtain these mutants.

TABLE 2
Insertion mutation in previously known virulence associated genes conserved among the selected bacteria

| Y. pestis | | | | | Growth | |
|---|---|---|---|---|---|---|
| Center | E. coli | | Yop | | rich media | Virulence |
| CDS number | Gene name | Function | secretion | Cytotoxicity | 26/37 degrees | (oral infection) |
| YPO2567 | phAc | Phosphate acetyltransferase | +++ | +++ | +++/++ | Attenuated |
| YPO0324 | uvrA | Exinuclease ABC subunit A | +++ | +++ | +++/+++ | Attenuated |
| YPO0372 | MiaA | tRNA-modification, miaA | +++ | +++ | +++/++ | Attenuated |
| YPO3097 | pta | Phosphoglucomutase, pta | +++ | +++ | +++/++ | Virulent |
| YPO3393 | pbp | Penicillin binding protein 1b, pbp | +++ | +++ | +++/+++ | Virulent |

TABLE 3
Deletion mutants

| | | | | | Oral |
|---|---|---|---|---|---|
| | | | | | attenuation |
| | | | Growth, rich | | Relative value |
| | Yop | Cytoxi- | media 26/37 | | (LD₅₀(Mutant)/ |
| Gene | secretion | city | degrees | LD₅₀ | LD₅₀(WT)) |
| vag1 | +++ | +++ | +++/+++ | 5.2 × 10⁹ | 104 |
| vag2 | +++ | +++ | +++/+++ | <7.4 × 10⁷ | 1 |
| vag3 | +++ | +++ | +++/+++ | 1.2 × 10⁹ | 24 |
| vag4 | nd | | | | |
| vag5 | +++ | +++ | +++/+++ | 1.8 × 10⁸ | 4 |
| vag6 | +++ | +++ | +++/+++ | 2.2 × 10⁸ | 4 |
| vag7 | +++ | +++ | +++/+++ | 1.6 × 10⁹ | 32 |
| vag8 | nd | | | | |
| vag9 | +++ | +++ | +++/+++ | 3.2 × 10⁹ | 64 |
| pta | +++ | +++ | +++/+++ | 1.6 × 10⁹ | 32 |
| MiaA | +++ | +++ | +++/+++ | >2 × 10⁹ | >100 |
| uvrC | +++ | +++ | +++/+++ | 1.4 × 10⁹ | 28 |

1. A method for identifying novel virulence associated genes, comprising:
selecting a pathogenic microorganism;
providing genetic information about said pathogenic microorganism comprising information about open reading frames (ORFs) having (a) assigned functions, (b) no assigned functions, (c) showing similarity with hypothetical proteins from other species;
selecting an obligate human parasite evolutionary deprived of a substantial portion of its coding capacity;
providing genetic information about said human parasite;
comparing the genetic information provided for said pathogenic microorganism with that provided for said obligate human parasite, thereby identifying at least one open reading frame (ORF) common to both coding for a protein (c) showing similarity with a hypothetical protein from other microorganism species.
introducing a mutation in said at least one ORF of said pathogenic organism to produce a mutated ORF;
assessing the pathogenicity of said mutated pathogenic organism;
comparing the pathogenicity of said mutated pathogenic organism with that of the corresponding non-mutated pathogenic organism.

2. The method of claim 1, wherein said substantial portion of coding capacity is 50% or more.

3. The method of claim 1, wherein said substantial portion of coding capacity is about 75%.

4. The method of claim 1, wherein said mutation is an insertion mutation.

5. The method of claim 1, wherein said mutation is a deletion mutation.

6. The method of claim 1, wherein said protein (c) is capable of substantially promoting in vivo growth of the microorganism by which it is expressed but is not essential for substantially promoting in vitro growth thereof in rich medium.

7. The method of claim 1, wherein said protein (c) is coded by a vag-gene conserved in said obligate parasite.

8. The method of claim 7, wherein the vag-gene is selected from vagA, vagC, vagG, vagH, and vagI.

9. The method of claim 1, wherein said obligate human parasite is selected from Yersinia pestis, Treponema pallidum, Neisseria gonorrhoeae, Helicobacter pylori, and Borrelia burgdorferi.

10. The method of claim 1, wherein said obligate human parasite is evolutionary closely related to said pathogenic microorganism.

11. A virulence-promoting protein expressed by the vag-gene of claim 8.

12. The protein of claim 11, comprising a single insertion or deletion mutation.

13. An antibody directed against the virulence-promoting protein of claim 11.

14. A monoclonal antibody directed against the virulence-promoting protein of claim 11.

15. A method of treating bacterial infection comprising administrating to a patient in need an effective amount of the antibody of claim 13.

16. A chemical agent, in particular an antibiotic, directed against the vag-gene of claim 7.

17. A chemical agent, in particular an antibiotic, directed against the vag-gene of claim 8.

18. A microorganism virulence reducing vaccine prepared from an attenuated vag-mutant strain.

19. The use of an attenuated vag-mutant strain as a vaccine carrier of heterologous antigens.

20. A method of treating an infection in a patient, comprising the administration of an effective amount of the chemical agent of claim 16

21. A method of treating an infection in a patient, comprising the administration of an effective amount of the chemical agent of claim 17.

22. A method of preventing an infection comprising the administration of the vaccine of claim 18.

23. A method of preventing an infection comprising the administration of the vaccine carrier of heterologous antigens of claim 19.

24. A method of treating bacterial infection in a patient, comprising the administration of an effective amount of the antibody of claim 13.

25. A method of treating bacterial infection in a patient, comprising the administration of an effective amount of the antibody of claim 14.

26. A method of treating bacterial infection comprising administrating to a patient in need an effective amount of the antibody of claim 14.
